(19) United States
(12) Patent Application Publication
(10) Pub. No.: US 2013/0090537 A1
(43) Pub. Date: Apr. 11, 2013

(54) BLOOD GLUCOSE SENSOR

(71) Applicants: Marcel F. Schemmann, Maria Hoop (NL); Thomas O'Brien, Eindhoven (NL)

(72) Inventors: Marcel F. Schemmann, Maria Hoop (NL); Thomas O'Brien, Eindhoven (NL)

(73) Assignee: 2M Engineering Limited, Veldhoven (NL)

(21) Appl. No.: 13/646,721

(22) Filed: Oct. 7, 2012

Related U.S. Application Data
(60) Provisional application No. 61/544,859, filed on Oct. 7, 2011.

(51) Int. Cl. A61B 5/1455

(52) U.S. Cl. 600/316

(57) ABSTRACT

A method to measure glucose within the blood of a tissue test area includes illuminating the tissue test area using a single mode light source at a point of incidence, with at least some of the light penetrating tissue at the point of incidence; calibrating the light source by adjusting a distance between the point of incidence and an axicon lens; collecting returning radiation from the tissue test area at a point offset from the point of incidence; removing tissue fluorescence using edge filters; removing additional tissue fluorescence by shifting the excitation wavelength of the single mode light source; heating the test area; and analyzing a returned Raman signal to determine the glucose within the blood.

### PRIORITY

This application claims priority under 35 U.S.C. 119 to USA provisional application No. 61/544,859 filed on Oct. 7, 2011, which is incorporated herein by reference in its entirety.

### BACKGROUND

Diabetes mellitus is a chronic condition in which the patient manifests a raised blood glucose concentration. This increased concentration is due to one or more of (1) lack of the hormone insulin, (2) a deficiency in the concentration of insulin, and (3) a deficiency in the level of insulin action.

Many serious conditions are associated with diabetes. These include premature coronary artery disease, blindness, renal failure and amputation. The two major types of diabetes are Type 1 and Type 2. Type 1 diabetes is cause by the destruction of the insulin producing B-cells in the pancreas. The B-cells are destroyed by the body's own immunogenic system. There are many causes of Type 2 diabetes, although the underlying mechanism in all causes is decreased insulin production. Obesity and physical inactivity are the most common cause of Type 2 diabetes. Type 2 diabetes is a progressive disease in that the production of insulin decreases slowly over several decades. Until recently, Type 2 diabetes was only diagnosed in adults, however it is now being diagnosed in children. Of the two major types of diabetes, Type 2 is by far the most common, present in approximately 90% of diabetics worldwide.

According to the World Health Organisation (WHO), there are more than 220 million people worldwide with diabetes. This figure is expected to rise to 300 million by 2025. In 2005 approximately 1.1 million people died worldwide from diabetes, with 80% being in low to middle income countries. The number of deaths due to diabetes is expected to rise by more than 50% by 2015, with an 80% increase in the number of deaths due to diabetes in middle to upper income countries.

Diabetes accounts for at least 5% of the total health care costs in European countries. This equates to £10 billion in the UK alone each year. The long term complications of diabetes account for 75% of this cost, with the remaining 25% being spent on diabetes management. In the USA in the late 1990s, the direct and indirect costs of diabetes amounted to $50 billion per year.

With careful blood glucose management, complications such as premature coronary artery disease, blindness, renal failure and amputations can all be avoided, leading to lower medical costs and prevented deterioration. Blood glucose management involves regular testing for the glucose levels in blood. One technique uses the finger stick method, whereby a drop of whole blood is extracted, placed on a stick sensor and the glucose level in the blood is measured. Ideally, diabetics should test their blood glucose levels at least four times a day. However diabetics generally test their blood glucose level on average only once a day. Reasons for this include, (1) the pain involved in the test, (2) dislike of the sight of blood, (3) cost, and (4) increased risk of infection.

### BRIEF DESCRIPTION OF THE DRAWINGS

FIG. 1 shows the bench-top device configuration with the probe attached for a 1^{st} embodiment of the invention.

FIG. 2 shows a detailed outline of the probe configuration.

FIG. 3 shows the Raman spectrum for glucose.

FIG. 4 shows the bench-top device configuration with the probe attached for a 2^{nd} embodiment of the invention.

FIG. 5 shows the bench-top device configuration with the probe attached for a 3^{rd} embodiment of the invention.

FIG. 6 shows a detailed outline of the probe configuration for a 4^{th} embodiment of the invention.

FIG. 7 shows the relative absorbance of various compounds vs light frequency.

FIG. 8 shows an example implementation with a laser source operating at a first wavelength and modulated by a frequency f1.

FIG. 9 illustrates a setup with a second laser modulated at a frequency f2, the Raman emission is coupled to the detector via the filter and the detector output is analyzed at a difference or du frequency of f1 and f2.

FIG. 10 shows coherent detection where an additional laser is tuned to the Raman frequency and mixed with the Raman signal.

FIG. 11 shows a single laser at lambda2 where part of the laser is split for detection of the Raman peak and another part is provided to the sample.

FIG. 12 shows scatter length as a function of wavelength.

### DESCRIPTION

Preliminaries

References to “one embodiment” or “an embodiment” do not necessarily refer to the same embodiment, although they may. Unless the context clearly requires otherwise, throughout the description and the claims, the words “comprise,” “comprising,” and the like are to be construed in an inclusive sense as opposed to an exclusive or exhaustive sense; that is to say, in the sense of “including, but not limited to.” Words using the singular or plural number also include the plural or singular number respectively, unless expressly limited to a single one or multiple ones. Additionally, the words “herein,” “above,” “below” and words of similar import, when used in this application, refer to this application as a whole and not to any particular portions of this application. When the claims use the word “or” in reference to a list of two or more items, that word covers all of the following interpretations of the word: any of the items in the list, all of the items in the list and any combination of the items in the list, unless expressly limited to one or the other.

“Logic” refers to machine memory circuits, machine readable media, and/or circuitry which by way of its material and/or material-energy configuration comprises control and/or procedural signals, and/or settings and values (such as resistance, impedance, capacitance, inductance, current/voltage ratings, etc.), that may be applied to influence the operation of a device. Magnetic media, electronic circuits, electrical and optical memory (both volatile and nonvolatile), and firmware are examples of logic.

Those skilled in the art will appreciate that logic may be distributed throughout one or more devices, and/or may be comprised of combinations memory, media, processing circuits and controllers, other circuits, and so on. Therefore, in the interest of clarity and correctness logic may not always be distinctly illustrated in drawings of devices and systems, although it is inherently present therein.

The techniques and procedures described herein may be implemented via logic distributed in one or more computing devices. The particular distribution and choice of logic is a design decision that will vary according to implementation.

Overview

The device described herein provides non-invasive (does not require breaking the skin) monitoring of blood glucose concentrations. The device is suitable for diabetics with Type 1 or Type 2 diabetes, as well as healthy people. The device includes a light source that emits light at two different wavelengths, optics for collecting the returning radiation, a detector and logic for analysing the returning radiation and determining the blood glucose level. The device collects the returning signal from a region away from the point of incidence. The device isolates the glucose Raman spectrum from the returning radiation via two methods. The first method utilizes the feature of the collecting optics blocking a majority of the tissue fluorescence. The second wavelength is only narrowly shifted from the first wavelength and the resulting returning radiation is compared to the original returning signal. The device has a moveable axicon lens that calibrates the device so that it always targets the blood in the capillary bed, regardless of the test site/skin thickness/tissue composition of the patient.

This device may be utilized for the detection and quantification of biological analytes based on Raman spectroscopy. It specifically detects and quantifies the glucose concentration within the blood stream. It can be used for continuous and semi continuous non-invasive measurement of the glucose concentration within the blood stream.

The temperature of the patient is monitored at the test site and Raman spectra are collected using two different wavelengths. Two different Raman spectroscopic methods, conventionally used exclusively of one another, are combined. These are Spatially Offset Raman Spectroscopy (SORS) and Shifted Excitation Raman Difference Spectroscopy (SERDS). Selective application of heat is also used despite the drawbacks conventionally understood to accompany the use of heat with Raman spectroscopy techniques.

SORS is a Raman spectroscopic method that is capable of eliciting Raman spectra from subsurface molecules without first eliminating the surface spectrum. SORS involves collecting Raman signals from the surface at a set distance removed from the point of incidence of the excitation light source. The further from the point of incident that the Raman spectrum is collected, the deeper it's point of origin within the media. The spectra collected at the point of incident are more likely to have been generated at, or very close to, the media surface. For homogenous media this isn't an issue, but for heterogeneous media, the surface spectra may mask the sub-surface spectra and will pollute the sub-surface spectra.

For wavelengths within the visible spectrum, the strong luminescence signal from tissue masks the majority of Raman bands and decreases the signal to noise (S/N) ratio of the measurements affecting directly the sensitivity and specificity values. In applications such as the analysis of biological specimens, laser exposure limits and long exposure times are an issue, and the luminescent background poses a significant problem to the routine use of Raman spectroscopy. At least two kinds of luminescence may be encountered in Raman spectroscopy, including fluorescence and phosphorescence. They may both be referred to as fluorescence, in spite of the physical origin. Due to its much longer lifetime, phosphorescence may be excited by light of shorter wavelengths, such as room light; the stored energy can be released later when stimulated by a longer wavelength laser beam. As a result, phosphorescence may contain a large portion of emission blue-shifted relative to the Raman excitation. Fluorescence has a lifetime longer than Raman but much shorter than phosphorescence and can be considered instantaneous on the scale of typical Raman integration times. It relies on excited electronic states and because fewer samples have chromophores excitable by light of longer wavelength, it is often less problematic when longer wavelength lasers are used. It is largely for this reason that lasers of near infrared wavelengths may be employed in Raman instruments, despite the disadvantage that Raman intensity also drops off as excitation wavelength increases.

SERD is a method which removes the luminescence from the recorded spectrum. By shifting the diode laser frequencies, the broad background remains approximately unchanged while the sharply peaked Raman bands follow the shifted excitation frequency. Subtraction of the two spectra obtained with slightly shifted excitation frequencies gives a derivative like spectrum from which the background has been effectively eliminated and Raman features can be extracted.

In some embodiments the temperature at the test site is increased. An increase in temperature leads to an increase in Raman signal. By increasing the temperature and utilising SORS and SERDS, the blood glucose concentration at the test site may be determined

Self calibration to differing skin thicknesses is achieved by altering the spatial offset in the probe and by monitoring a Raman peak at approximately 2200 cm⁻¹. This is the peak from the C═N bond and indicates that the returning radiation is coming from a region rich in haemoglobin.

### Description of Various Embodiments

### Embodiment 1

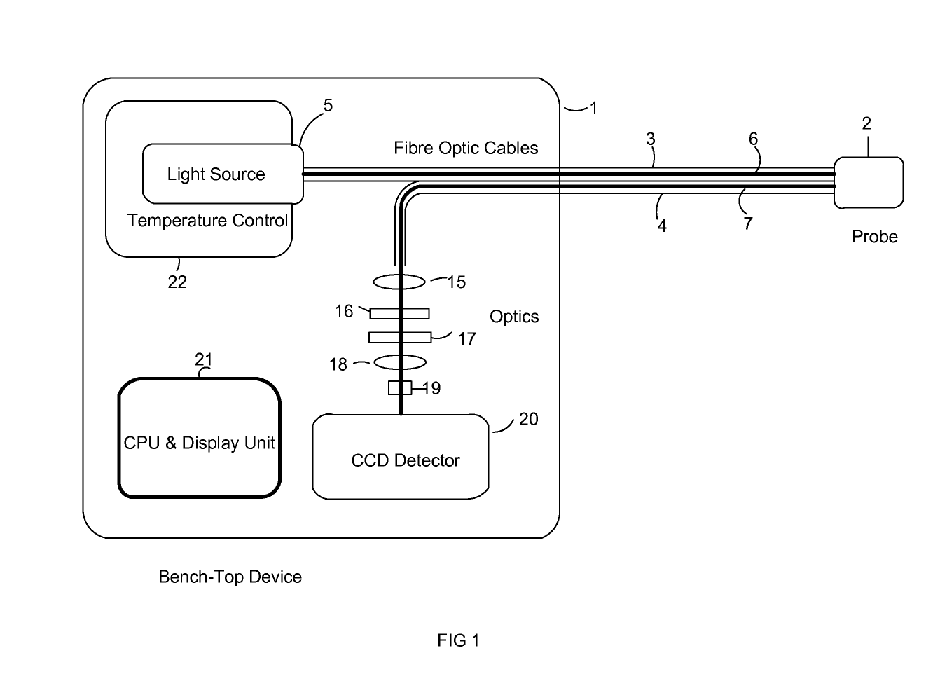

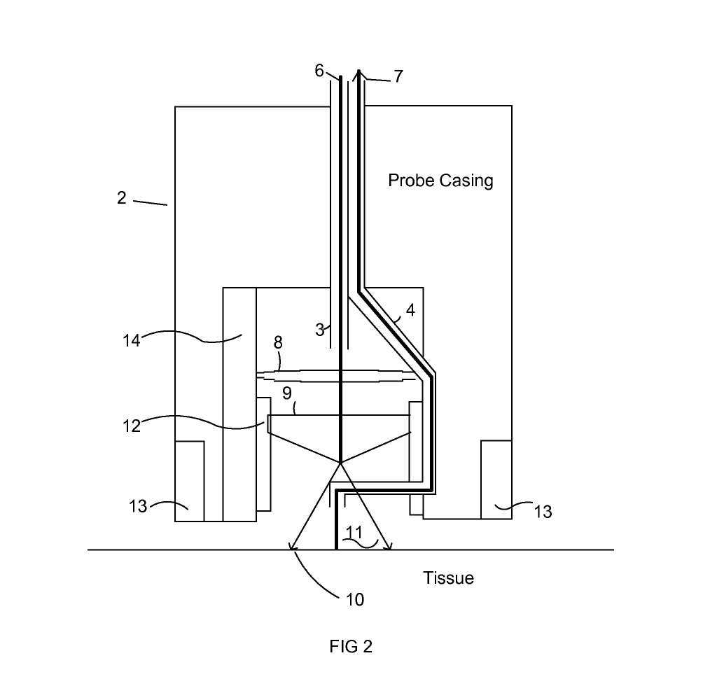

A probe 2 is connected to the bench-top component 1 via fibre optic cables 3 and 4, as shown in FIG. 1. The light source 5 within the table top component is a 670 nm single mode laser. The incident ray 6 travels from the 670 nm laser 5 to the probe 2 through a fibre optic cable 3. Within the probe 2, as shown in FIG. 2, the incident ray 6 passes through a convex lens 8, forming a parallel beam, then through an axicon lens 9 which results in the incident ray forming a ring before it strikes the test site. The returning radiation 7 is collected by a fibre optic cable 4 at an offset distance 11 from the point of incidence 10. This offset distance 11 can be altered by moving the axicon lens mounting 12 closer or further away from the point of incidence 10. Returning radiation 7 collected at a site offset 11 from the point of incidence 10 indicates an origin not from the tissue surface, but instead the sub-surface. The probe also has a temperature probe 14 and a heating element 13, both of which are controlled by the Central Processing Unit (CPU) 21.

The returning radiation 7 is collected by a fibre optic cable 4 and travels back to the table top component. It passes through a convex lens 15 which results in a parallel beam being formed. It then passes through an edge filter 16 that only allows radiation with a wavelength of 780 nm or greater through. This removes any light of the same wavelength as the incident light 6. This edge filter 16 also blocks the majority of tissue fluorescence. The 2^{nd} edge filter 17 only allows radiation of wavelength less than 850 nm through. This removes the Raman signal from water and narrowly defines the region over which the Raman spectra are being collected. It also ensures that only radiation of wavelength less than 1000 nm hits the silicon based CCD detector 20. After the radiation has passed through the 2^{nd} edge filter 17, it then passes through another convex lens 18 which focusses the radiation onto a collimating lens 19. The collimating lens 19 focusses the beam as a very narrow parallel beam on to the CCD detector 20.

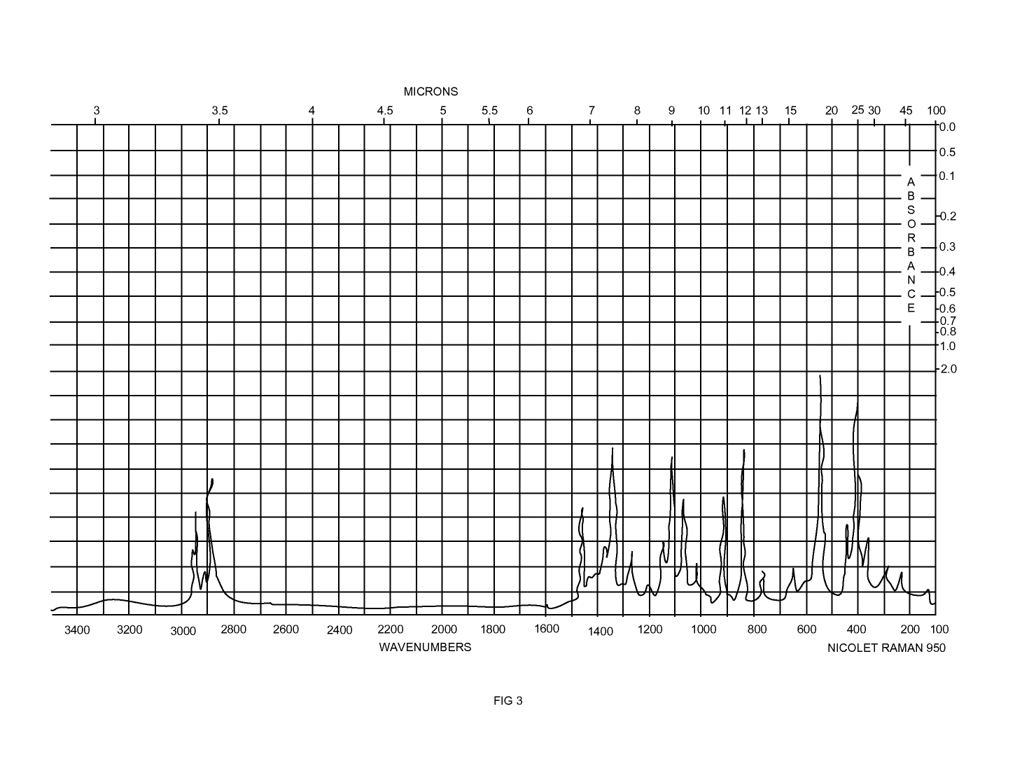

The Central Processing Unit (CPU) 21 then causes storage of the spectrum detected by the CCD detector 20. A temperature control unit 22 surrounds the 670 nm laser 5. This is controlled by the CPU 21 and keeps the temperature of the laser 5 steady. Once a spectrum has been recorded, the temperature control unit 22 increases the temperature of the 670 nm laser 5. This results in a shift in wavelength of the incident ray 6 to 670.5 nm. By slightly shifting the laser 5 wavelength, the broad background remains approximately unchanged while the sharply peaked Raman bands, shown in FIG. 3, follow the shifted excitation frequency. Subtraction of the two spectra obtained with slightly shifted excitation frequencies gives a derivative like spectrum from which the background has been effectively eliminated and Raman features can be extracted. A Raman spectrum for glucose is recorded and logic compares it to the original spectrum recorded. Any remaining tissue fluorescence is removed in this manner and the logic identifies the peaks that result from glucose.

The heating element 13 then locally raises the tissue temperature at the site by a set amount. This is monitored by the temperature probe 14. An increase in temperature results in a measureable increase in Raman signal. Another Raman spectrum is collected using light of wavelength 670.5 nm. This is then compared to the two original spectra. The glucose peaks in the region between 785 nm and 850 nm are analysed and from this the glucose concentration is determined.

The target is the glucose dissolved in the blood stream. This device calibrates to ensure that it is targetting the blood stream within the capillary bed, by altering the distance the axicon lens 9 is from the surface of the skin. The closer it is to the surface of the skin, the smaller the spatial offset 11, the closer to the surface that the radiation 7 is being collected from. The further from the point of incident 10 that the radiation 7 is collected, the deeper it's point of origin within the media.

For calibration in respect to skin thickness at the test site, increase the spatial offset 11 from its minimum until the CCD detector 20 detects a large peak at approximately 2200 cm⁻¹. This is the peak from the C═N bond and indicates that the returning radiation 7 is coming from a region rich in haemoglobin. This region should be the capillary bed which will be rich in blood, and thus haemoglobin.

### Embodiment 2

In an alternative procedure for measuring the blood glucose concentration, within the probe the axicon lens 9 is positioned at its lowest level, resulting in the smallest spatial offset 11. A Raman spectrum is recorded at this position. This is the Raman spectrum for the skin of the patient at the particular test site. The axicon lens housing 12 then increases the spatial offset between the radiation collection fibre 4 and the point of incidence 10 incrementally, recording spectra until a peak at approximately 2200 cm⁻¹ is detected, signifying that the Raman signal is coming from the blood stream. In this manner the device is self-calibrating. This spectrum is cleaned by subtracting the skin Raman spectrum to ensure that the skin is not having an effect on the blood Raman spectrum. From this cleaned blood spectrum, the glucose peaks are identified and recorded.

The Central Processing Unit (CPU) 21 then stores this spectrum. Once a spectrum has been recorded, the temperature control unit 22 increases the temperature of the 670 nm laser 5. This results in a shift in wavelength of the incident ray 6 to 670.5 nm. By slightly shifting the laser 5 wavelength, the broad background remains approximately unchanged while the sharply peaked Raman bands, shown in FIG. 3, follow the shifted excitation frequency. Subtraction of the two spectra obtained with slightly shifted excitation frequencies gives a derivative like spectrum from which the background fluorescence has been effectively eliminated and this further cleans the blood Raman spectrum. The glucose peaks are again identified and recorded.

The heating element 13 then locally raises the tissue temperature at the site by a set amount. This is monitored by the temperature probe 14. An increase in temperature results in a measureable increase in Raman signal. Another Raman spectrum is collected using light of wavelength 670.5 nm. This is then compared to the blood spectrum. The glucose peaks in the region between 785 nm and 850 nm are analysed and, taking into account the increase in Raman signal due to the increase in temperature, the glucose concentration is determined.

### Embodiment 3

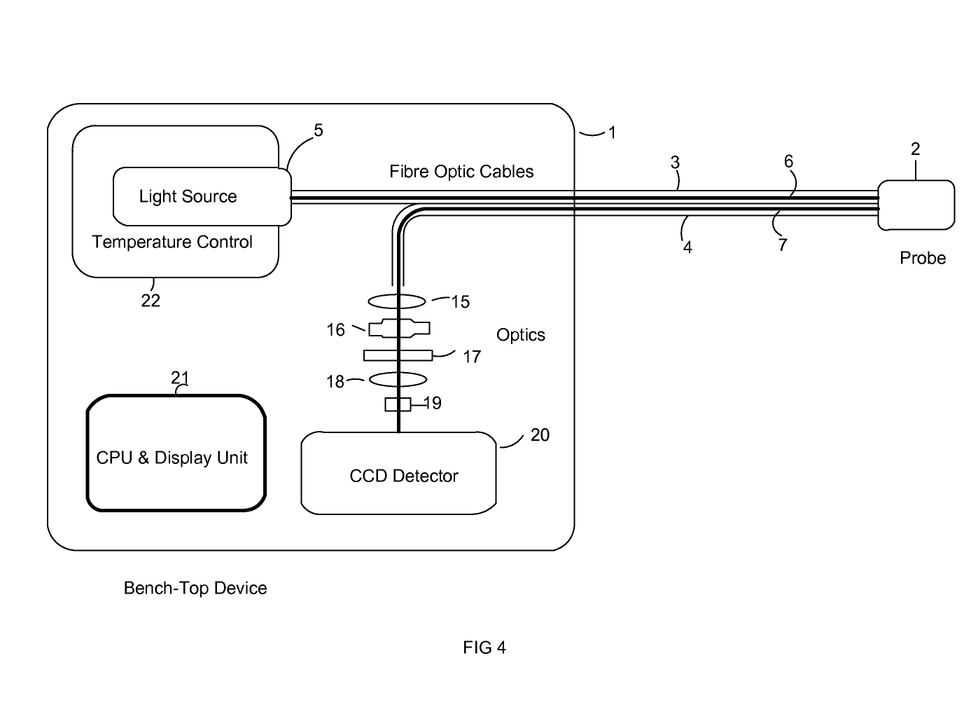

See FIG. 2, FIG. 3, and FIG. 4. In this embodiment the bench-top device has a rotating stage 16 that allows the replacement of the 780 nm edge filter with a 800 nm edge filter. The CPU controls the rotating stage 16. The 780 nm edge filter only allows transmission of radiation of 780 nm in wavelength or greater, while the 800 nm edge filter only allows transmission of radiation of 800 nm in wavelength or greater.

Using the 670 nm laser 5, the device collects a Raman spectrum with the 780 nm edge filter 16 in place and with the minimum possible spatial offset 11. This is the Raman spectrum for the skin at the particular test site. The device then calibrates itself with regards to skin thickness by altering the increase the spatial offset 11 from its minimum until the CCD detector 20 detects a large peak at approximately 2200 cm⁻¹. This is the peak from the C═N bond and indicates that the returning radiation 7 is coming from a region rich in haemoglobin. This region should be the capillary bed which will be rich in blood, and thus haemoglobin. For this calibration step, the filter that is in place in the rotating stage 16 is the 780 nm edge filter. Once calibration has been accomplished, this filter is removed and replaced by the 800 nm edge filter. This has effect of removing more of the tissue fluorescence from the returning radiation 7 than the 780 nm edge filter.

A Raman spectrum is then recorded at this position. This spectrum is cleaned by subtracting the skin Raman spectrum to ensure that the skin is not having an effect on the blood Raman spectrum. From this cleaned blood spectrum, the glucose peaks are identified and recorded.

The Central Processing Unit (CPU) 21 then stores this spectrum. Once a spectrum has been recorded, the temperature control unit 22 increases the temperature of the 670 nm laser 5. This results in a shift in wavelength of the incident ray 6 to 670.5 nm. By slightly shifting the laser 5 wavelength, the broad background remains approximately unchanged while the sharply peaked Raman bands, shown in FIG. 3, follow the shifted excitation frequency. Subtraction of the two spectra obtained with slightly shifted excitation frequencies gives a derivative like spectrum from which the background fluorescence has been effectively eliminated and this further cleans the blood Raman spectrum. The glucose peaks are again identified and recorded.

The heating element 13 then locally raises the tissue temperature at the site by a set amount. This is monitored by the temperature probe 14. An increase in temperature results in a measureable increase in Raman signal. Another Raman spectrum is collected using light of wavelength 670.5 nm. This is then compared to the blood spectrum. The glucose peaks in the region between 785 nm and 850 nm are analysed and, taking into account the increase in Raman signal due to the increase in temperature, the glucose concentration is determined.

### Embodiment 4

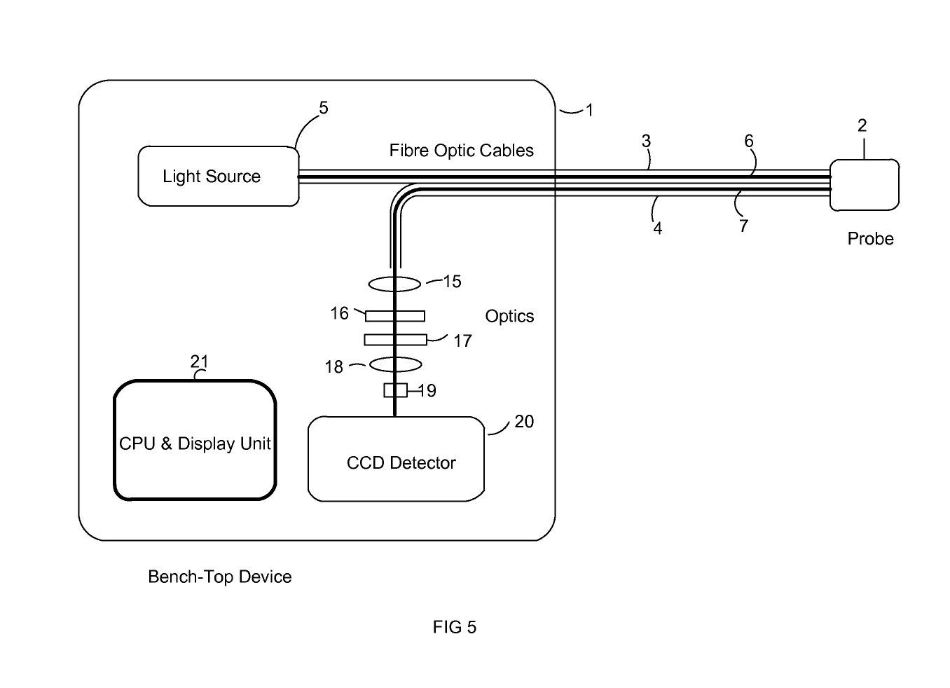

See FIG. 2, FIG. 3, and FIG. 5. This differs from the preferred embodiment in that the device utilises temperature increase and Spatially Offset Raman Spectroscopy (SORS) to determine the blood glucose concentration. It does not utilise Shifted Excitation Raman Difference Spectroscopy (SERDS) in this embodiment.

Using the 670 nm single mode laser 5, the device collects a Raman spectrum with the minimum possible spatial offset 11. This is the Raman spectrum for the skin at the particular test site. The device then calibrates itself with regards to skin thickness by altering the increase the spatial offset 11 from its minimum until the CCD detector 20 detects a large peak at approximately 2200 cm⁻¹. This is the peak from the C═N bond and indicates that the returning radiation 7 is coming from a region rich in haemoglobin. This region should be the capillary bed which will be rich in blood, and thus haemoglobin.

A Raman spectrum is then recorded at this position. This spectrum is cleaned by subtracting the skin Raman spectrum to ensure that the skin is not having an effect on the blood Raman spectrum. From this cleaned blood spectrum, the glucose peaks are identified and recorded.

The Central Processing Unit (CPU) 21 then stores this spectrum. The heating element 13 then locally raises the tissue temperature at the site by a set amount. This is monitored by the temperature probe 14. An increase in temperature results in a measureable increase in Raman signal. Another Raman spectrum is collected using light of wavelength 670.5 nm. This is then compared to the blood spectrum. The glucose peaks in the region between 800 nm and 850 nm are analysed and, taking into account the increase in Raman signal due to the increase in temperature, the glucose concentration is determined.

### Embodiment 5

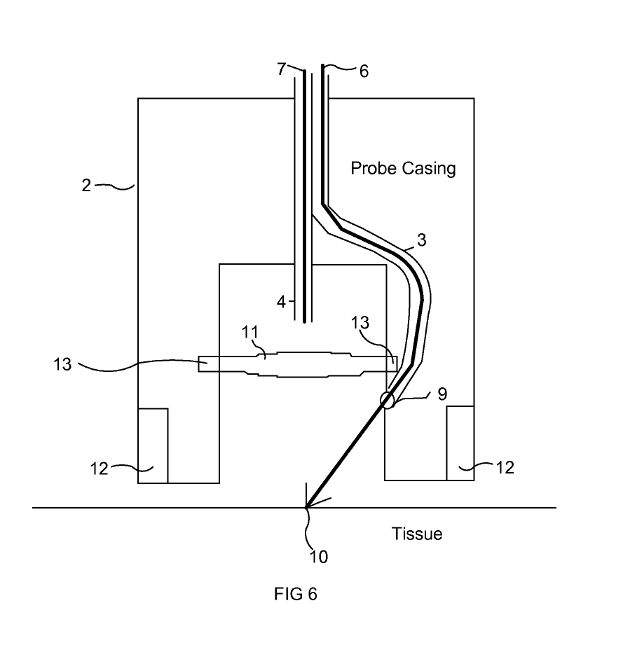

See FIG. 1, FIG. 3, and FIG. 6. In this embodiment the device utilises temperature increase and Shifted Excitation Raman Difference Spectroscopy (SERDS) to determine the blood glucose concentration. It does not utilise Spatially Offset Raman Spectroscopy (SORS) in this embodiment. It is not self-calibrating with regards to skin thickness.

The device includes of a bench-top component 1 and a probe 2 connected to the bench-top component via fibre optic cables 3 and 4, as shown in FIG. 1. The light source 5 within the table top component is a 670 nm single mode laser. The incident ray 6 travels from the 670 nm laser 5 to the probe 2 through a fibre optic cable 3. Within the probe 2, as shown in FIG. 6, the incident ray 6 passes through a ball lens 9, focussing the beam onto the test site 10. An aspheric lens collects the returning radiation 7 from the point of incidence 10, and directs it into a fibre optic cable 4. The aspheric lens is fixed in a mounting 13.

The returning radiation 7 is collected by a fibre optic cable 4 and travels back to the table top component. It passes through a convex lens 15 which results in a parallel beam being formed. It then passes through an edge filter 16 that only allows radiation with a wavelength of 800 nm or greater through. This removes any light of the same wavelength as the incident light 6. This edge filter 16 also blocks the majority of tissue fluorescence. The 2^{nd} edge filter 17 only allows radiation of wavelength less than 850 nm through. This removes the Raman signal from water and narrowly defines the region over which the Raman spectra are being collected. It also ensures that only radiation of wavelength less than 1000 nm hits the silicon based CCD detector 20. After the radiation has passed through the 2^{nd} edge filter 17, it then passes through another convex lens 18 which focusses the radiation onto a collimating lens 19. The collimating lens 19 focusses the beam as a very narrow parallel beam on to the CCD detector 20.

The Central Processing Unit (CPU) 21 then stores (causes to be stored) the spectrum detected by the CCD detector 20. There is a temperature control unit 22 surrounding the 670 nm laser 5. This is controlled by the CPU 21 and it keeps the temperature of the laser 5 steady. Once a spectrum has been recorded, the temperature control unit 22 increases the temperature of the 670 nm laser 5. This results in a shift in wavelength of the incident ray 6 to 670.5 nm. By slightly shifting the laser 5 wavelength, the broad background remains approximately unchanged while the sharply peaked Raman bands, shown in FIG. 3, follow the shifted excitation frequency. Subtraction of the two spectra obtained with slightly shifted excitation frequencies gives a derivative like spectrum from which the background has been effectively eliminated and Raman features can be extracted. A Raman spectrum for glucose is recorded and logic compares it to the original spectrum recorded. Any remaining tissue fluorescence is removed in this manner and the logic identifies the peaks that result from glucose.

The heating element 13 then locally raises the tissue temperature at the site by a set amount. This is monitored by the temperature probe 14. An increase in temperature results in a measureable increase in Raman signal. Another Raman spectrum is collected using light of wavelength 670.5 nm. This is then compared to the two original spectra. The glucose peaks in the region between 800 nm and 850 nm are analysed and from this the glucose concentration is determined

Alternate Approach to Distinguishing Raman Spectrum

The Raman spectrum needs to be distinguished from the fluorescence and phosphorescence spectra. Manners of doing this have been discussed involving shifted Raman spectroscopy and proper choice of wavelengths, with detection based on proper filter selection and long detector integration times.

Here an alternate approach is introduced. The timescale of phosphorescence is very long, exceeding the ms (millisecond) timescale. The timescale for relevant fluorescence effects is 0.2 nsec (nanoseconds, 800 MHz cutoff frequency) to several nsec (less than 100 MHz cutoff frequency) where most of the fluorescence occurs with time constants in the several nsec range. The spontaneous emission and the optical gain due to Raman scattering occur on a much shorter timescale.

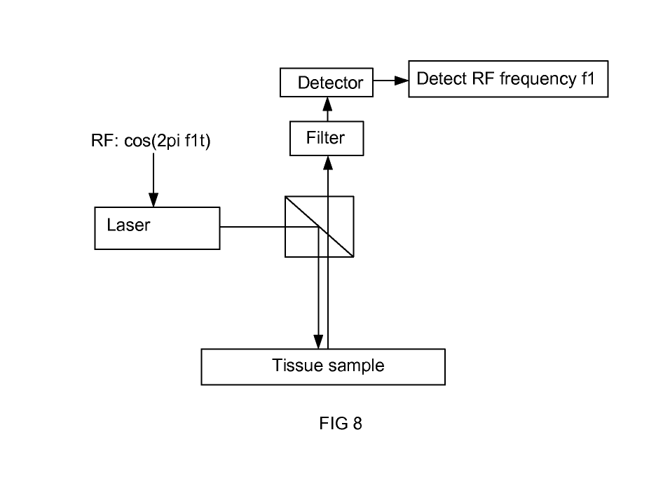

A modulated excitation wavelength may be used, with modulation frequencies that exceed these cutoff frequencies. By choosing a modulation frequency in excess of 100 MHz, most of the fluorescence spectrum has a reduced modulation response, whereas the Raman spectrum will instantaneously track the modulation. With a high-speed detector, the modulation response may be determined as the RF (Radio Frequency, for instance a frequency greater than 100 MHz) output of the detector. The response of the fluorescence at the detector output is reduced by choosing a suitably high modulation frequency. This frequency is preferably chosen above 1 GHz. FIG. 8 illustrates an example implementation with a laser source operating at a first wavelength and modulated by RF frequency 1. The laser is coupled to the tissue of interest and the Raman emission from the tissue is collected and provided to a detector via a filter that selects a Raman peak of interest. The detector output at the modulation frequency is analyzed.

The Raman spectrum is due to spontaneous emission; however there is also Raman gain where the gain spectrum is similarly shaped as the spontaneous emission spectrum. When an excitation (pump) laser is modulated with a frequency f1 then the Raman gain is modulated by that same frequency. If a probe laser is directed at a sample then the loss (or gain) of the probe light will be modulated by that same frequency f1 such that the probe beam will be modulated by a process known as Stimulated Raman Scattering (SRS). The Raman gain is due to molecular vibrations such that the impulse of photons can be accommodated in the molecular vibration. As a result the SRS may produce photons that are directed in a different direction than the incoming photons (net change of impulse for incoming and outgoing photons), including in the reverse direction. Thus a reflection of the probe may be found modulated with frequency f1. This reflected pump light may be detected using filters or using coherent detection means. The probe may in principle also cause “stimulated” fluorescence so that frequency f1 should still be set high enough. The directivity of this stimulated emission however is in the direction of the probe beam, generally not in reverse.

The probe itself may also be modulated with a second frequency f2. The SRS will then contain frequency components at f1, f2, f1+f2 and f1−f2 and dc. Stimulated emission from states that contribute to fluorescence may also contain each of the frequencies. However if f1 and f2 are chosen high enough then the fluorescence contribution will only contribute a dc term. The frequency component at f1−f2 is particularly suitable to detect as it can be chosen arbitrarily low by selecting a small difference between f1 and f2. Thus a low frequency (such as 10 MHz) can be chosen for f1−f2 which will permit low-speed detectors for picking up the Raman signal or even a camera for suitably low choice of f1−f2. Such a low frequency detector will average out the high frequency components at f1, f2 and f1+f2. An advantage of using low-speed detectors is that the area of such detectors can generally be chosen larger such that it is easier to collect enough light for detection. In case a camera is used and the frequency f1−f2 is chosen suitably, preferably such that f1−f2 is half the frame rate, then comparison of subsequent images for instance by computing the intensity difference provides an image of the pump modulation by the Raman process.

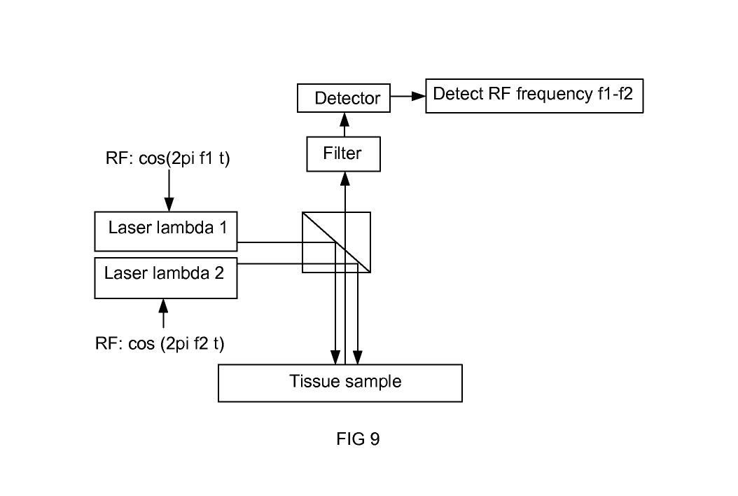

FIG. 9 illustrates a setup with a second laser modulated at a frequency f2, the Raman emission is coupled to the detector via the filter and the detector output is analyzed at a difference or du frequency of f1 and f2. In this case the filter need not be a sharp filter as the wavelength of the probe (second) laser determines at which wavelength the Raman gain is probed. The filter may even be omitted but can be useful to reject unwanted light from wavelengths that are not of interest.

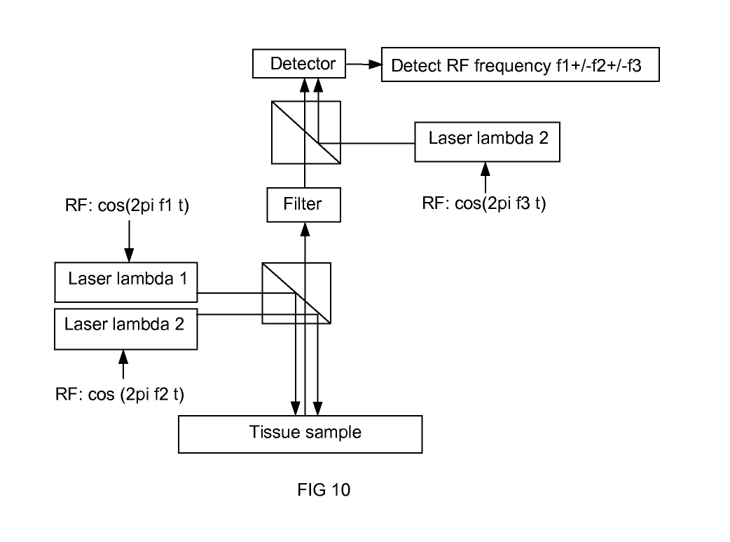

Filters can be used to detect the Raman spectrum. An alternate method is to use coherent detection. Coherent detection permits operation above the thermal noise floor of a detector such that the obtainable signal to noise ratio is limited by shot noise and laser RIN only. This is helpful for detecting high frequency modulated signals in weak signals. This is illustrated in FIG. 10 where an additional laser is tuned to the Raman frequency and mixed with the Raman signal. The additional laser may be modulated at a third frequency f3 and any sum or difference of these frequencies may be used at the analysis of the detector output.

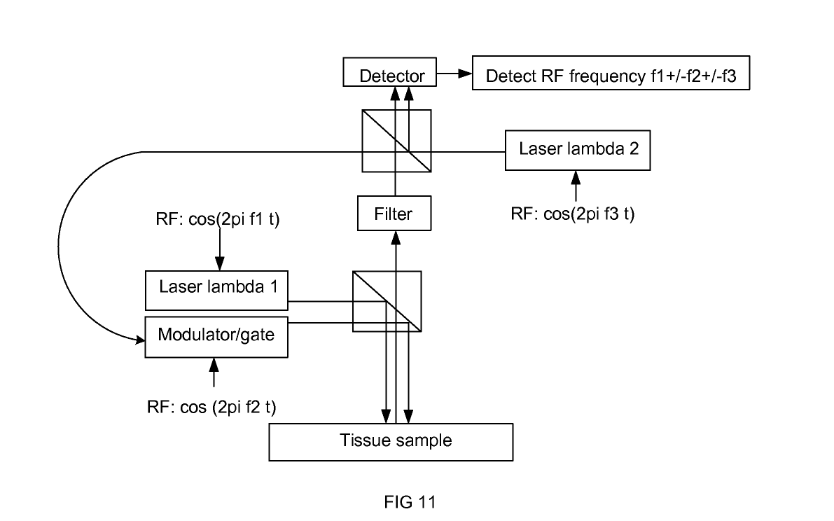

A drawback of the system shown in FIG. 10 is that it is hard to tune two lasers precisely to the same wavelength (lambda 2). If they are not exactly tuned to the same wavelength a beat frequency is generated at the detector equal to the optical frequency difference of the two lasers. While this beat frequency may be used on purpose it can be easier to use a single laser at lambda2 where part of the laser is split for detection of the Raman peak and another part is provided to the sample. This second part can optionally but need not be provided to a modulator that can add an additional modulation frequency to the second part. This is illustrated in FIG. 11.

The wavelength of the pump and probe can be shifted relative to each other such that the Raman spectrum can be scanned. When the difference in wave number between pump and probe equals to a wave number where a peak occurs in the Raman spectrum then the detected signal at frequency f1−f2 will be strong and the Raman spectrum as shown in FIG. 3 can be reproduced. Shifting of both pump and probe is permitted such that the difference in wave number of pump and probe can be shifted over a wide range.

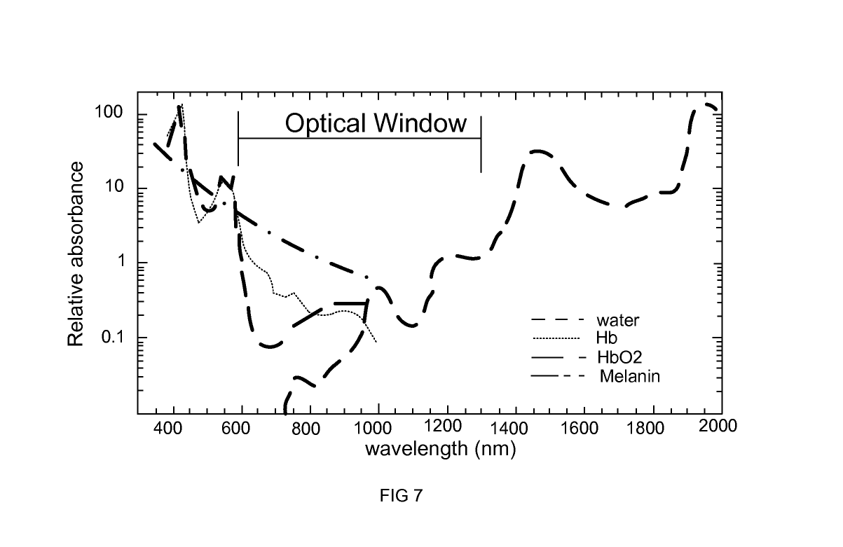

An example implementation can be made using DFB lasers with wavelengths in a range commonly used in the CWDM communication bands. A pump wavelength of 1271.5 nm may be chosen and combined with probe wavelengths as 1300 and 1330 nm. The 1300 nm wavelength is used for glucose detection of Raman peaks at wave number shifts of 1030, 1070 and 1120 nm respectively by tuning the probe laser over 2.5 nm. With a typical wavelength sensitivity of 0.08 nm/deg. C. that implies that about 30 degree C. temperature tuning of the probe laser is used. Alternately the pump and probe are both shifted by 15 deg. C. each. The 1330 nm laser is used for haemoglobin detection at a wave number shift of 2200. The haemoglobin detection is used to locate blood vessels. In a preferred implementation both 1300 and 1330 nm probes may be operated simultaneously but with different frequencies f2 and f3 respectively at each wavelength. Thus different frequencies will be generated by Raman processes where f1−f2 corresponds to pump-probe interaction with the 1300 nm laser for glucose detection and f1−f3 provides simultaneous haemoglobin detection from pump-probe interaction with the 1330 nm laser. There can also be an f2−f3 frequency generated by interaction of the two probe lasers that may be used for further analysis or filtered out of the detector output signal. The wavelengths are still within the optical transmission window that is shown in FIG. 7 depicting the per-cm absorption of water and relative absorption for melatonin and haemoglobin (with and without oxygen saturation). The maximum wavelength used still has absorption on the order of 1/cm such that it will not be a problem to have several mm of tissue thickness in the measurement. The presence of absorption does however affect the absolute signal level and the amount of blood that participates in the measurement is also unknown. For this reason it is preferred to use the ratio between detected glucose and haemoglobin Raman signal levels as a measure for the amount of glucose per unit blood.

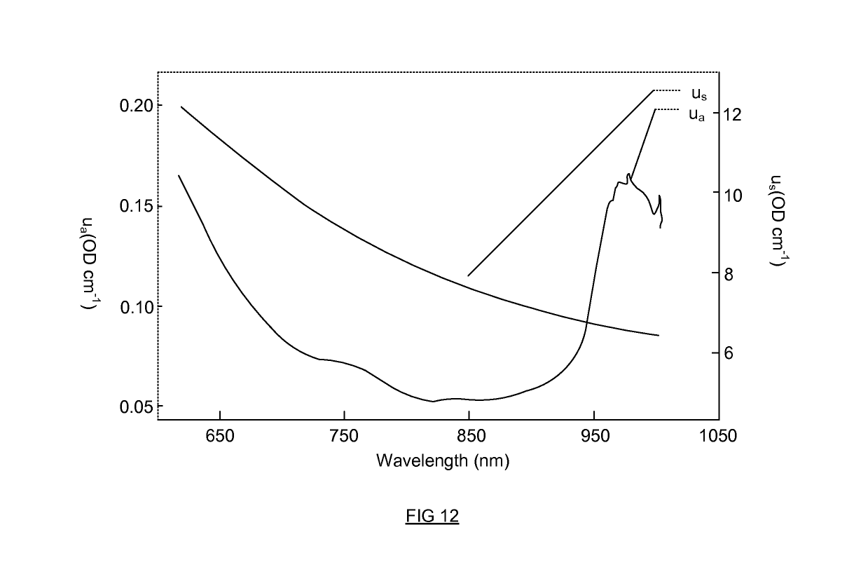

The use of a pump and a probe laser can offer the advantage of defining a region of intersection of pump and probe lasers, for instance at a depth under the skin, by defining a region where the beams overlap by focussing and directing beams into that region. The use of long wavelength lasers such as lasers in the 1300 nm region offers the advantage that the scatter length (uₛ curve in FIG. 12) is reduced when compared to shorter wavelengths such at 600 nm. As a result pump and probe beams can penetrate deeper into skin without becoming scattered excessively and thus by directing two, optionally focussed, beams at each other under an angle a point of intersection can be defined at some depth under the skin. This depth is preferably chosen to coincide with the expected location of blood vessels or other tissue of interest.

Use of pump and probe lasers with Raman scattering leads to a modulation of the probe laser as has been discussed. Now it should be noted that any probe laser modulation is due to the transfer of photons from the pump laser (higher photon energy) to the probe laser (lower energy) through a molecular vibration that makes up for the energy and impulse difference. Thus the pump laser is depleted of photons in this process and a modulation will also be present in the pump laser. Thus detection of the Raman scattering will also be visible as a modulation of the remaining pump light. This light is scattered in tissue and part of it can be collected. Thus in all the applications with pump and probe lasers the detection of Raman scattering can also be based on collected pump light from the tissue or both. Reversing pump and probe role can be of interest to reduce the fluorescence which will be weaker at photon energies exceeding the probe energy (which is lower than the pump). It can also be of interest in implementations where multiple probe wavelengths exist that are used to interact with a single pump and only one set of detector optics will be needed for that pump. Those optics could for instance include a homodyne coherent detection system as illustrated in FIG. 11 that may be easiest to implement for just a single wavelength.

Implementations and Alternatives

The techniques and procedures described herein may be implemented via logic distributed in one or more computing devices. The particular distribution and choice of logic is a design decision that will vary according to implementation.

Those having skill in the art will appreciate that there are various logic implementations by which processes and/or systems described herein can be effected (e.g., hardware, software, and/or firmware), and that the preferred vehicle will vary with the context in which the processes are deployed. “Software” refers to logic that may be readily readapted to different purposes (e.g. read/write volatile or nonvolatile memory or media). “Firmware” refers to logic embodied as read-only memories and/or media. Hardware refers to logic embodied as analog and/or digital circuits. If an implementer determines that speed and accuracy are paramount, the implementer may opt for a hardware and/or firmware vehicle; alternatively, if flexibility is paramount, the implementer may opt for a solely software implementation; or, yet again alternatively, the implementer may opt for some combination of hardware, software, and/or firmware. Hence, there are several possible vehicles by which the processes described herein may be effected, none of which is inherently superior to the other in that any vehicle to be utilized is a choice dependent upon the context in which the vehicle will be deployed and the specific concerns (e.g., speed, flexibility, or predictability) of the implementer, any of which may vary. Those skilled in the art will recognize that optical aspects of implementations may involve optically-oriented hardware, software, and or firmware.

The foregoing detailed description has set forth various embodiments of the devices and/or processes via the use of block diagrams, flowcharts, and/or examples. Insofar as such block diagrams, flowcharts, and/or examples contain one or more functions and/or operations, it will be understood as notorious by those within the art that each function and/or operation within such block diagrams, flowcharts, or examples can be implemented, individually and/or collectively, by a wide range of hardware, software, firmware, or virtually any combination thereof. Several portions of the subject matter described herein may be implemented via Application Specific Integrated Circuits (ASICs), Field Programmable Gate Arrays (FPGAs), digital signal processors (DSPs), or other integrated formats. However, those skilled in the art will recognize that some aspects of the embodiments disclosed herein, in whole or in part, can be equivalently implemented in standard integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and/or firmware would be well within the skill of one of skill in the art in light of this disclosure. In addition, those skilled in the art will appreciate that the mechanisms of the subject matter described herein are capable of being distributed as a program product in a variety of forms, and that an illustrative embodiment of the subject matter described herein applies equally regardless of the particular type of signal bearing media used to actually carry out the distribution. Examples of a signal bearing media include, but are not limited to, the following: recordable type media such as floppy disks, hard disk drives, CD ROMs, digital tape, and computer memory.

In a general sense, those skilled in the art will recognize that the various aspects described herein which can be implemented, individually and/or collectively, by a wide range of hardware, software, firmware, or any combination thereof can be viewed as being composed of various types of “circuitry.” Consequently, as used herein “circuitry” includes, but is not limited to, electrical circuitry having at least one discrete electrical circuit, electrical circuitry having at least one integrated circuit, electrical circuitry having at least one application specific integrated circuit, circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), circuitry forming a memory device (e.g., forms of random access memory), and/or circuitry forming a communications device (e.g., a modem, communications switch, or optical-electrical equipment).

Those skilled in the art will recognize that it is common within the art to describe devices and/or processes in the fashion set forth herein, and thereafter use standard engineering practices to integrate such described devices and/or processes into larger systems. That is, at least a portion of the devices and/or processes described herein can be integrated into a network processing system via a reasonable amount of experimentation.

The foregoing described aspects depict different components contained within, or connected with, different other components. It is to be understood that such depicted architectures are merely exemplary, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected”, or “operably coupled”, to each other to achieve the desired functionality.

What is claimed is:

1. A method to measure glucose within the blood of a tissue test area, comprising :
illuminating the tissue test area using a single mode light source at a point of incidence, with at least some of the light penetrating tissue at the point of incidence;
calibrating the light source by adjusting a distance between the point of incidence and an axicon lens;
collecting returning radiation from the tissue test area at a point offset from the point of incidence;
removing tissue fluorescence using edge filters;
removing additional tissue fluorescence by shifting the excitation wavelength of the single mode light source;
heating the test area; and
analyzing a returned Raman signal to determine the glucose within the blood.

2. The method of claim 1, wherein Raman spectroscopy is used to collect a Raman spectrum from the tissue test area.

3. The method of claim 1 wherein Spatially Offset Raman Spectroscopy is used to calibrate a penetration depth of light at the point of incidence.

4. The method of claim 1 wherein Shift Excitation Raman Difference Spectroscopy is used to remove fluorescence from the test area at the point of incidence.

5. The method of claim 1 wherein light in the visible range is a primary wavelength output by the single mode light source.

6. The method of claim 5 where the light source is a single mode laser.

7. The method of claim 6 where an excitation wavelength of the single mode laser is 670 nm.

8. The method of claim 1 wherein a heating element is used to heat the light source to increase an excitation wavelength of the light source by 0.5 nm.

9. The method of claim 1 wherein a Raman spectrum from the test area is collected using excitation light of wavelength 670 nm.

10. The method of claim 1 wherein a Raman spectrum from the test area is collected using excitation light of wavelength 670.5 nm.

11. The method of claim 1 wherein a Raman spectrum from the test area is collected using excitation light of wavelength 670.5 nm after the test area has been heated locally.

12. The method of claim 1 wherein a position of the axicon lens relative to the test area is altered vertically for the calibration.

13. The method of claim 3 wherein a Raman return signal for haemoglobin is detected to determine that incident light has reached the targeted blood vessel at the test area.
